(19) United States
(12) Patent Application Publication
(10) Pub. No.: US 2018/0053396 A1
(43) Pub. Date: Feb. 22, 2018

(54) System and Method for Identifying the Presence of Moisture

(71) Applicant: Morton Greene, Las Vegas, NV (US)

(72) Inventor: Morton Greene, Las Vegas, NV (US)

(21) Appl. No.: 15/683,245

(22) Filed: Aug. 22, 2017

Related U.S. Application Data
(60) Provisional application No. 62/377,976, filed on Aug. 22, 2016.

(51) Int. Cl. G08B 21/20; G06F 19/00; A61F 13/42; A61F 13/00

(52) U.S. Cl. CPC G08B 21/20; G06F 19/3418; H04W 4/22; A61F 13/00055; A61F 2013/424; A61F 13/42

(57) ABSTRACT

A device confirms the presence of liquid via signals received from a sensor. The device provides for the confirmation of the presence of liquid in a sleeping or resting environment. The detection and alerting of unintended liquid present in a sleeping environment alerts a third party to the presence of such liquid by remote means. The device is further enabled to detect a liquid pattern change over time.

### CROSS REFERENCE TO RELATED APPLICATIONS

This application claims priority to U.S. Provisional 62/377,976, filed Aug. 22, 2016, and U.S. Provisional 62/415,789, filed Nov. 1, 2016, which are hereby incorporated by reference as if submitted in their entireties.

### FIELD OF THE INVENTION

This invention relates to the field of moisture detection and more particularly, to a system and method for identifying the presence of moisture. And the reporting of volume captured or contained from time to time.

### BACKGROUND OF THE INVENTION

The presence of moisture in a sleeping environment may cause many unintended health and safety issues. Common moisture issues related to sleeping environments include urination and other incidents of body fluids and liquids, such as those associated with bile, which may or may not be captured by a diaper. Although any person may experience on episode of bedwetting, for example, younger and older individuals may experience the unintended incidents of liquid on a more frequent basis.

### SUMMARY OF THE INVENTION

A system and method in the field of moisture detection and more particularly, to a system and method for identifying the presence of moisture, is disclosed.

Embodiments of the invention provide for a device which confirms the presence of liquid. Embodiments of the invention provide for the confirmation of the presence of liquid in a sleeping environment. Embodiments of the invention provide for the detection and alerting of unintended liquid present in a sleeping or resting environment and may, particularly, alert a third party to the presence of such liquid by remote means.

Embodiments of the invention provide for the detection of a liquid pattern over a certain time period. As liquid spreads over a certain area of an absorbent pad, such as a diaper, a pattern may be determined and further analyzed.

### BRIEF DESCRIPTION OF THE FIGURES

Understanding of the present invention will be facilitated by consideration of the following detailed description of the preferred embodiments of the present invention taken in conjunction with the accompanying drawings, in which like numerals refer to like parts:

FIG. 1 illustrates an exemplary device in accordance with at least one embodiment of the present invention;

FIG. 2 illustrates another exemplary device in accordance with at least one embodiment of the present invention;

FIG. 3 illustrates a sensory environment in accordance with at least one embodiment of the present invention; and

FIG. 4 illustrates an exemplary computing environment in accordance with at least one embodiment of the present invention.

### DETAILED DESCRIPTION OF THE PREFERRED EMBODIMENTS

It is to be understood that the figures and descriptions of the present invention have been simplified to illustrate elements that are relevant for a clear understanding of the present invention, while eliminating, for the purpose of clarity, many other elements found in typical document processing systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

The repeated use of antibiotics, beginning with newborns, easily allows the development of resistances to antibiotics and create serious problems with diseases later in their life. Each day a small amount of Diaper Rash Ointment is applied to the skin of an infant. These added antibiotics added to the product, pathogens gradually develop resistance to the antibiotics added to the ointment. The present invention may reduce the need to apply medicated ointment.

Diaper rash may occur when skin is in contact with a wet diaper for long periods of time, infrequently changed diapers, diarrhea, and the use of plastic pants to enclose diapers thus creating high moisture levels within the covered area. This may result in painful bacterial or yeast (fungal) infection for the little infant. A rather common form of inflamed skin (dermatitis). Diaper rashes can occur intermittently, anytime an infant wears diapers, but they're most common in babies during their first 15 months, peaking between 9 and 12 months of age.

Rashes may begin as a simple skin infection that may spread to the surrounding region. The area covered by a diaper, buttocks, thighs and genitals are especially vulnerable because it's warm and moist, making a perfect breeding ground for bacteria and yeast. These rashes can be found within the creases of the skin, and there may be red dots scattered around the creases.

Many disposable diapers have added, Super Absorbent Polymers, (SAP), which will hold water away from the skin, and when touched the diaper pad feels dry. The parents or others checking for wetness, are misled into believing the pad is dry to the touch, but this Polymer called SAP does not stop the ammonia vapors or feces to irritate the skin without direct contact.

Delay of timely diaper change for infants is a major cause for acidic reactions of the perianal skin and urinary tract, resulting in maceration (softening and whitening of skin due to extended moisture contact) leading to a variety of infections. Treatments for these infections greatly increase financial expenses for parents, and are generally associated with a significant decrease in an infant's comfort and quality of life.

Urine has an additional impact on skin integrity because of its effect on skin pH. Studies show that ammonia alone is a skin irritant, when urea breaks down in the presence of fecal urease it increases pH caused by the ammonia released, this in turn promotes the activity of fecal enzymes. These fecal enzymes increase the skin's hydration and permeability to bile salts which also act as skin irritants.

An answer for the diaper rash problem is to prevent diaper rash from occurring, thus reducing the need to apply medicated ointment frequently. The present invention was designed to reduce the incidences of painful and more serious skin complications through an active infant void alerting technology. The present invention employs a novel active, non-invasive monitoring system that significantly enhances baby care. The product is designed to minimize incidences of serious skin complications through the active detection of moisture captured in an infant or adult diaper and other absorbent bed or mattress protection materials.

An embodiment of the present invention, inclusive of the smart diaper alert system and sensor, is a way we care for our precious infants from their beginning with their birth.

The present invention may provide new parents a solution to end the problems of infections, medication resistance and of course the dirty diapers can be changed long before they become, smelly. The system and sensor of the present invention may transmit an alert long before the smelly alert to change a diaper becomes apparent.

With the millennial generation coming of age the current worldwide fertility rate is now estimated at 4.2 children per thousand in population yearly. This is creating increased demands on families, hospitals and care facilities.

The present invention may take advantage of this growth market with a dedicated and experienced team providing, effective management and marketing skills needed for success.

In an embodiment of the present invention, one of the system and/or sensor of the present invention may be used through a subscription which may begin with, for example, shipping a first supply of a portion of the system. Similarly, for example, a first one month supply pack of disposable diapers communicative with the present invention.

The present invention may utilize an Internet of Things strategy which may be based on advertising to current and potential new customers to make them familiar about the products and the subscription service availability through an embodiment of the present invention. This may be accomplished through implementing a market advertising campaign that will ensure that we are known and respected in the new infant market. In an embodiment of the present invention, a subscription price may take into consideration the cost of production and distribution so as to ensure that we remain viable and profitable.

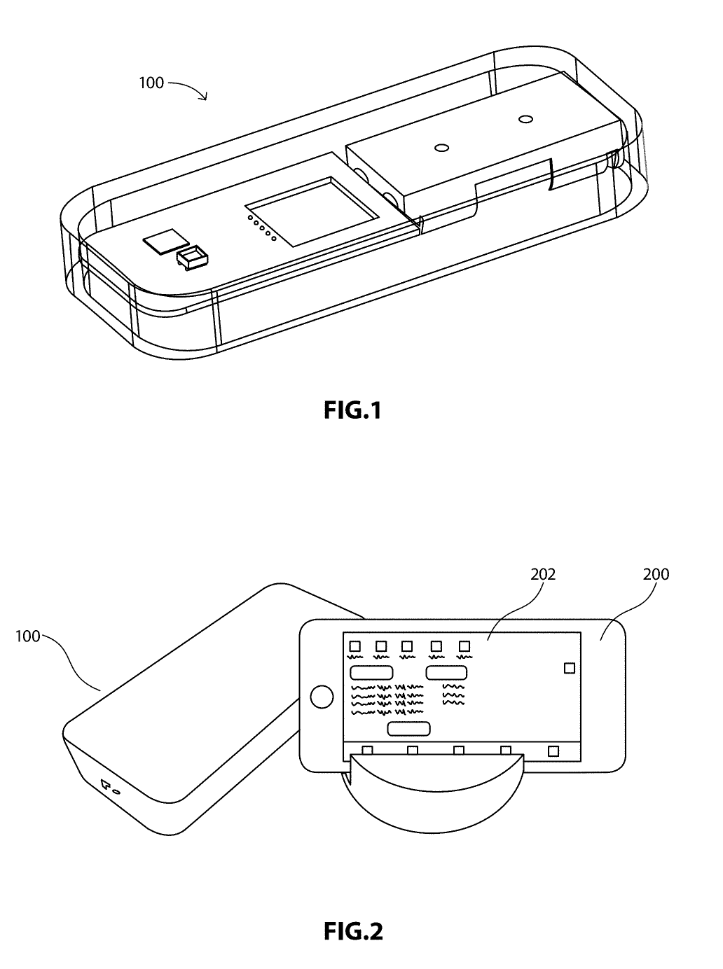

In an embodiment of the present invention with respect to FIG. 1, a system sensor 100 may be placed under a crib mattress and will send an alert message moments after the baby's incident, a message for a parent or caregiver to change the disposable diaper. Nano reactive materials may be added with the liquid absorbent materials in diapers along with SAP to absorb the liquid in seconds to prevent leaks. Disposable diaper and bed pad changing procedures remain the same. The sensor links to a smart diaper in a matter of seconds.

In an embodiment of the present invention, a sensor 100 may be placed under a stroller seat using an adhesive, such as Velcro®, supplied with the subscriber's initial order shipment. The sensor and pouch may be easily removable. The sensor may automatically recognize and adjust system sensitivity for the new stroller material and environment. As with the crib mattress sensor will monitor the diaper and text a signal when soiling occurs.

In an embodiment of the present invention, a sensor pocket may be placed at the bottom of a baby pouch and held in place with an adhesive type seat pocket. This optional component can be ordered at the time of subscription or at a later time. It can also be used with chest carry packs. Also it can be used as Car Seat Sensor (See #4) below.

In an embodiment of the present invention, a sensor may be placed at the bottom of the baby seat and held in place with Velcro®, for example It may also be used with chest carry packs. It may auto activate when the baby is within 5 inches (12 cm) from the sensor and may alert the smart phone of the user of the system when the baby has soiled the diaper equipped with the system.

In an embodiment of the present invention, the system may read or compute the distance and speed of separation between your baby seat's sensor and a smart phone. At a predetermine distance, such as 20 ft, for example, an alert message may be sent to the user's smart phone that your baby is in the car, for example. At 30 ft and a separation speed of 2 ft per second, for example, it may send a warning that your baby is in the car alone and the system will alert the police of an emergency “Baby Alone In Car” and provide both car GPS and the smart phone location (where possible).

A liquid pattern over a certain time period may be detected and be analyzed upon. As liquid spreads over a certain area of an absorbent pad, such as a diaper, a pattern may be determined. Further analysis of the liquid pattern may be done to determine liquid amount, such as in milliliters, temperature, and/or viscosity.

The present invention may connect to an automobile's computer system, and may work with any car, any model and any year since 1980, and even earlier. For example, a sensor may simply attach to a car seat with an adhesive Velcro® strip.

In an embodiment of the present invention, and as shown in FIG. 2 with respect to device 200, subscribers may use an app 202 associated with the system of the present invention which may measure the distance and separation speed between your smart phone and the a “Smart Diaper Sensor” in the car seat. For example, in the event the separation speed between the phone and sensor is greater than 7 miles per hour the system software will assume that a person is stealing the car, maybe with your baby in the back seat. This may automatically trigger a signal module to prepare the text settings for a 911 police emergency call. There is an additional optional sensor module which has GPS built in which will continuously transmit the location coordinates to enable the GPS module to report physical location every 3 (few) minutes. It can also work with your (a) stroller, in the park or most other outdoor locations.

In an embodiment of the present invention, a smart sensor may be purchased as an independent unit and may be used without diaper sensing features. Such a unit may not sense the baby soiling the sensor enabled diaper.

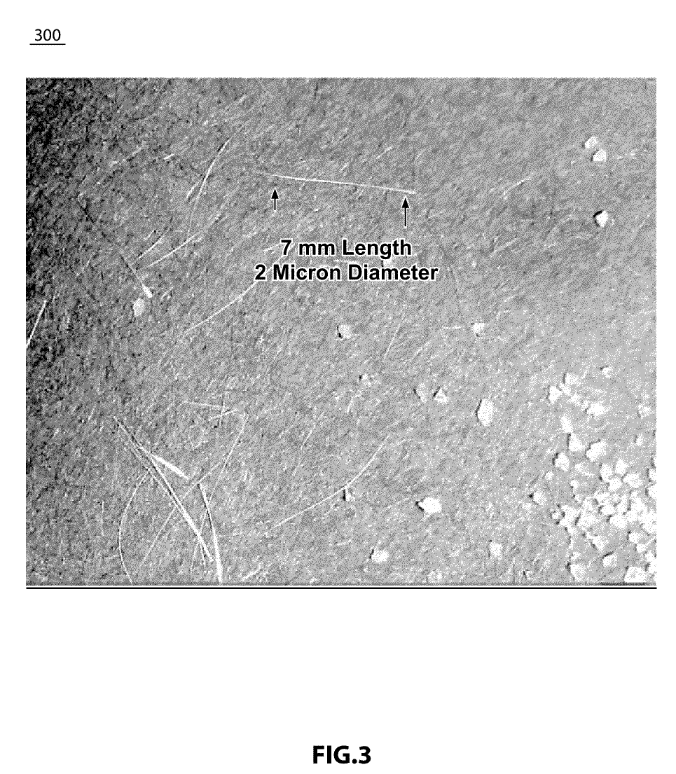

With respect to FIG. 3, each pad 300 may be individually sterilized and sealed in a sealed sterile plastic bag. This may protect the baby from possible exposure to other germs which may be lurking in the material. The sensing feature may include Nano EM (Electromagnetic) reactive materials that may be added with the liquid absorbent materials in diapers along with SAP to absorb the liquid in seconds to prevent leaks. The sensing feature may be 7 mm in length and 2 Micron in diameter, for example. (Sensors of device 100 may use NFC radar to identify the presence of moisture already in use by many (Automatic Windshield Wipers)). For example, a first picture or snapshot of the sensing features may be taken. After a certain period of time, for example 5-10 seconds, a subsequent picture or snapshot may be taken to detect changes, such as a change in pattern of EM reflections. In response to detected change, appropriate alerts may be transmitted. Alerts may include, but are not limited to, e-mail, text messages, in-app messages, or the like, based on user-set preferences. Further parameters may be determined based on collected data by the device sensors. For example, volume output (in milliliters), temperature, or even pH. In the event that multiple sensors are used (i.e. twin babies), sensing features, or resonators, of one diaper may differ from another diaper. For example, resonators material may be spaced apart at different density levels such as placing a certain amount of resonator fiber dipoles per square inch.

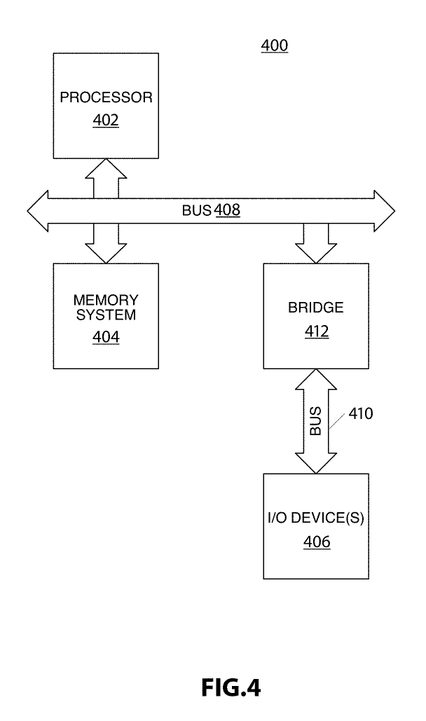

FIG. 4 is an example of a simplified functional block diagram of a computer system 400. The functional descriptions of the present invention can be implemented in hardware, software or some combination thereof.

As shown in FIG. 4, the computer system 400 includes a processor 402, a memory system 404 and one or more input/output (I/O) devices 406 in communication by a communication ‘fabric’. The communication fabric can be implemented in a variety of ways and may include one or more computer buses 408, 410 and/or bridge and/or router devices 412 as shown in FIG. 4. The I/O devices 406 can include network adapters and/or mass storage devices from which the computer system 400 can send and receive data for generating and transmitting alerts. The computer system 400 may be in communication with the Internet or other devices using conventional protocols via the I/O devices 408.

Those of ordinary skill in the art will recognize that many modifications and variations of the present invention may be implemented without departing from the spirit or scope of the invention. Thus, it is intended that the present invention cover the modification and variations of this invention provided they come within the scope of the appended claims and their equivalents.

The various illustrative logics, logical blocks, modules, and engines, described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but, in the alternative, the processor may be any conventional processor, controller, microcontroller, or state of the art system machine. A processor may also be implemented as a combination of computing devices, e.g., a combination of a DSP and a microprocessor, a plurality of microprocessors, one or more microprocessors in conjunction with a DSP core, or any other such configuration.

Further, the steps and/or actions of a method or algorithm described in connection with the aspects disclosed herein may be embodied directly in hardware, in a software module executed by a processor, or in a combination of the two. A software module may reside in RAM memory, flash memory, ROM memory, EPROM memory, EEPROM memory, registers, a hard disk, a removable disk, a CD-ROM, or any other form of storage medium known in the art. An exemplary storage medium may be coupled to the processor, such that the processor can read information from, and write information to, the storage medium. In the alternative, the storage medium may be integral to the processor. Further, in some aspects, the processor and the storage medium may reside in an ASIC. Additionally, the ASIC may reside in a user terminal. In the alternative, the processor and the storage medium may reside as discrete components in a user terminal. Additionally, in some aspects, the steps and/or actions of a method or algorithm may reside as one or any combination or set of instructions on a machine readable medium and/or computer readable medium.

Those of ordinary skill in the art may recognize that many modifications and variations of the present invention may be implemented without departing from the spirit or scope of the invention. Thus, it is intended that the present invention covers the modifications and variations of this invention provided they come within the scope of the appended claims and their equivalents.

I claim:

1. A method for identifying moisture by a device, the method comprising:
by one or more sensors, detecting the presence of moisture via a sensing feature;
generating a first alert; and
transmitting the first alert to a remote device.

2. The method of claim 1, wherein the sensing feature has a responsive feature and is implanted in a diaper or pad absorbent material layer.

3. The method of claim 1, where the one or more sensors automatically recognizes and adjusts system sensitivity based on new environment placement.

4. The method of claim 3, wherein the new environment is under a crib, under a bed, near a car seat, or in a stroller.

5. The method of claim 1, wherein the alert is sent via text message.

6. The method of claim 1, further comprising:
calculating distance and separation speed between the device and the remote device;
generating a second alert in response to the distance and separation speed reaching a first threshold;
sending the second alert to the remote device.

7. The method of claim 6, further comprising:
generating a third alert in response to the distance and separation speed reaching a second threshold;
sending the third alert to a third party.

8. The method of claim 7, wherein the third alert is a 911 police emergency call.

9. The method of claim 1, wherein the sensing feature comprises nano reactive or resonant materials.

10. The method of claim 1, wherein the remote device senses puss filling an absorbent layer of a bandage.

11. A system for identifying moisture by a device, the system comprising:
one or more sensors configured to:
detect the presence of moisture via a sensing feature;
generate a first alert; and
transmit the first alert to a remote device.

12. The system of claim 11, wherein the sensing feature has a responsive feature and is implanted in a diaper or pad absorbent material layer.

13. The system of claim 11, where the one or more sensors automatically recognizes and adjusts system sensitivity based on new environment placement.

14. The system of claim 13, wherein the new environment is under a crib, under a bed, near a car seat, or in a stroller.

15. The system of claim 11, wherein the alert is sent via text message.

16. The system of claim 11, further comprising:
calculating distance and separation speed between the device and the remote device;
generating a second alert in response to the distance and separation speed reaching a first threshold;
sending the second alert to the remote device.

17. The system of claim 16, further comprising:
generating a third alert in response to the distance and separation speed reaching a second threshold;
sending the third alert to a third party.

18. The system of claim 17, wherein the third alert is a 911 police emergency call.

19. The system of claim 11, wherein the sensing feature comprises nano reactive or resonant materials.

20. The system of claim 11, wherein the remote device senses puss filling an absorbent layer of a bandage.
